(19) United States
(12) Patent Application Publication
(10) Pub. No.: US 2022/0110525 A1
(43) Pub. Date: Apr. 14, 2022

(54) INTERLEAVED LIGHT SOURCES AND METHODS OF THEIR USE

(71) Applicant: SpectraWAVE, Inc., Bedford, MA (US)

(72) Inventors: Martin F. Seifert, New Hartford, CT (US); James E. Muller, Auburndale, MA (US)

(73) Assignee: SpectraWAVE, Inc., Bedford, MA (US)

(21) Appl. No.: 17/298,219

(22) PCT Filed: Nov. 26, 2019
(86) PCT No.: PCT/US19/63390

§ 371 Date: May 28, 2021

Related U.S. Application Data
(60) Provisional application No. 62/774,161, filed on Nov. 30, 2018.

(51) Int. Cl. A61B 5/00

(52) U.S. Cl. CPC A61B 5/0066; A61B 5/0086; A61B 5/0075

(57) ABSTRACT

Described herein are systems with interleaved light sources and methods of their use. In some embodiments, a system includes a plurality of light sources, each of which has a bright phase and a dark phase, the system being arranged and constructed so that, for a first light source and a second light source of the plurality, the bright phase of the first light source occurs during the dark phase of a second light source. The example method may further include providing light from the second light source during a dark phase of the first light source. A first and/or second light source may be a swept source or a broadband source. A first and/or second light source may be a laser.

### PRIORITY APPLICATION

The present application claims the benefit of U.S. Provisional Patent Application No. 62/774,161, filed on Nov. 30, 2018, the content of which is hereby incorporated by reference herein in its entirety.

### BACKGROUND

Light sources are frequently used to characterize samples (e.g., materials). For example, one class of techniques for sample characterization is optical tomography, such as optical coherence tomography (OCT). Optical tomography techniques allow an image (e.g., tomographic reconstruction) of a sample to be generated. Another example of a class of techniques for sample characterization is spectroscopy. Some spectroscopic techniques use light to analyze composition of a material. For example, near-infrared spectroscopy (NIRS) is commonly used for sample characterization. In many cases, it is desirable to perform imaging and composition analysis for the same area of a material to develop a full understanding of the material. As various characterization techniques require using light of different wavelengths in different ways, it is typical to perform data collection for each characterization technique in a sequential manner. Data collection conventionally involves running one or more scans for a sample area for one technique followed by one or more scans for the sample area for another technique.

### SUMMARY

Sequential scanning is inefficient in many circumstances. It can take a long time, at least relatively, to scan a sample using one laser (e.g., thereby performing a tomographic scan) and then scan another sample using another laser (e.g., thereby performing a spectroscopic analysis). Such slowness can be a major detriment in certain applications. For example, during a catheter imaging procedure, it is particularly desirable to rapidly scan a patient. There is a need therefore for systems and methods that reduce sample characterization time when multiple techniques are being used. The present disclosure includes the recognition that interleaving light sources by alternately providing light from one source and then from another (e.g., in a rapidly alternating manner) can reduce sample characterization time.

The present disclosure provides an additional insight that in certain circumstances, such as measurements inside a human coronary artery, additionally or alternatively to long scanning times for multiple characterization techniques, it can be difficult to be certain that the multiple characterization techniques each acquire signal from substantially the same location. The present disclosure identifies the source of this co-registration problem (e.g., sequential scanning) and provides a solution for minimizing it, for example by acquisition of multiple signals with a single probe and interleaved light sources (e.g., that rapidly alternate between their bright phase and dark phase).

Characterization time and/or co-registration error can be reduced by interleaving light sources. Described herein are systems, apparatus, and/or methods that use interleaved light sources for providing electromagnetic radiation. In typical characterization techniques, there is some period of time during which illumination light is provided and some period where it is not. The period where source light is absent can be, for example, a period in which light from a sample is detected or a light source is reset in some way (e.g., a swept source is cycled back to an initial state). The period of time during which illumination light is not being provided can be used to provide illumination light for a second characterization technique. By providing illumination from a first light source for a period of time (a bright phase) that is during a period of time in which a second light source is not providing light (a dark phase), total characterization time for a sample and/or co-registration error may be reduced. The light provided by the first light source and/or second light source may be near-infrared light, for example used in NIRS or OCT.

In certain applications, a large sample is characterized using multiple techniques in discrete sections. For example, when characterizing using two techniques, each technique may be performed over a subset area (or volume) of the sample area (or volume) to be characterized. Such is the case in typical catheter imaging applications where a catheter is pulled back through, for example, an artery. A probe rotates rapidly (sometimes in excess of 10,000 rpm) as the catheter is pulled back and light is provided to characterize the artery. Speed of pullback is essential because imaging is possible only during seconds of time (e.g., ˜3 seconds) in which blood is removed from the artery by a flush, for example of contrast material or saline. Therefore, in some embodiments, it is desirable to maximize data collection, while minimizing co-registration error, over a period of time as the period for data collection may be limited by extrinsic factors. Even short reductions in time to provide necessary illumination for a plurality of techniques can reduce the time needed to properly characterize each discrete portion of a sample and, therefore, have a large impact on overall sample characterization time. In applications like catheterization procedures, reducing time to characterize a sample (e.g., artery) is beneficial (e.g., because it reduces the likelihood of complications from the procedure). Moreover, maintaining accurate co-registration of multiple signals is additionally or alternatively beneficial in certain applications, like catheterization procedures for example.

In some aspects, this disclosure provides a system comprising: a plurality of light sources, each of which has a bright phase and a dark phase, the system being arranged and constructed so that, for a first light source and a second light source of the plurality of light sources, the bright phase of the first light source occurs during the dark phase of the second light source. In some embodiments, the system is arranged and constructed so that the bright phase of the second light source occurs during the dark phase of the first light source. In some embodiments, the first light source and/or the second light source is a swept source. In some embodiments, the first light source and/or the second light source is a broadband source.

In some embodiments, a system is arranged and constructed so that a bright phase of a first light source is coincident with a dark phase of a second light source and a dark phase of the first light source is coincident with a bright phase of the second light source.

In some embodiments, at least one of a first light source and a second light source is a swept source. In some embodiments, a dark phase for at least one of a first light source and a second light source comprises a cycle phase. In some embodiments, a dark phase for at least one of a first light source and a second light source is a cycle phase.

In some embodiments, a light source is a swept source (e.g., a swept source laser) that is constructed and arranged to emit light in a wavelength band comprising (e.g., centered around) a characterization peak for characterizing arterial plaque (e.g., by sweeping through the band). In some embodiments, a light source is a broadband source that is constructed and arranged to emit light in a wavelength band comprising (e.g., centered around) a characterization peak for characterizing arterial plaque.

In some embodiments, a system comprises a common probe for a first light source and a second light source, the common probe comprising an optical fiber.

In some embodiments, a system comprises an optical coherence tomography (OCT) subsystem comprising an OCT detector and a second light source. In some embodiments, the system comprises a near-infrared spectroscopy (NIRS) subsystem comprising a NIRS detector and a first light source that is interleaved with the second light source.

In some embodiments, a plurality of light sources comprises a third light source, wherein the system comprising the plurality of light sources is arranged and constructed so that a dark phase of a first light source and a dark phase of a second light source are partially coincident, defining a common dark period for the first light source and the second light source, and a bright phase of the third light source occurs during the common dark period.

In some embodiments, a system is constructed and arranged so that (i) alternation between a bright phase and a dark phase of a first light source occurs at a first light source alternation frequency of at least 10 Hz (e.g., and no more than 500 kHz), (ii) alternation between a bright phase and a dark phase of a second light source occurs at a second light source alternation frequency of at least 10 Hz (e.g., and no more than 500 kHz), or (iii) both (i) and (ii). In some embodiments, the first light source alternation frequency is at least 10 kHz (e.g., and no more than 10 GHz) and the second light source alternation frequency is at least 10 kHz (e.g., and no more than 10 GHz).

In some aspects, the present disclosure provides a method comprising providing (e.g., emitting) first light (e.g., to a sample) from a first light source during a dark phase of a second light source. In some embodiments, the method further comprises providing (e.g., emitting) second light (e.g., to the sample) from the second light source during a dark phase of the first light source. In some embodiments, the first light source provides the first light throughout the dark phase of the second light source. In some embodiments, the second light source provides the second light throughout the dark phase of the first light source.

In some embodiments, a method comprises cycling a second light source during a dark phase of the second light source. In some embodiments, a method comprises cycling a first light source during a dark phase of the first light source.

In some embodiments, a method comprises receiving, via a first detector, a first signal generated, at least in part, using first light from a first light source. In some embodiments, the first signal is an optical coherence tomography (OCT) signal. In some embodiments, the method comprises receiving, via a second detector, a second signal generated, at least in part, using second light from a second light source. In some embodiments, the second signal is a near-infrared spectroscopy (NIRS) signal.

In some embodiments, a method comprises providing (e.g., emitting) third light from a third light source during a dark phase for a first light source and a dark phase for a second light source.

In some aspects, the present disclosure provides a system for characterizing a sample, the system comprising: a plurality of light sources, each of which has a bright phase and a dark phase, the system being arranged and constructed so that, for a first light source and a second light source of the plurality of light sources, the bright phase of the first light source occurs during the dark phase of the second light source, wherein each light source is for providing light for a characterization technique.

In some aspects, the present disclosure provides a method for characterizing a sample, the method comprising providing (e.g., emitting) first light to a sample from a first light source during a dark phase of a second light source, wherein light provided from the first light source is used in a first characterization technique and light provided from the second light source is used in a second characterization technique.

In some aspects, the present disclosure provides a system for treating a sample, the system comprising: a plurality of light sources, each of which has a bright phase and a dark phase, the system being arranged and constructed so that, for a first light source and a second light source of the plurality of light sources, the bright phase of the first light source occurs during the dark phase of the second light source, wherein at least one of the plurality of light sources is operable to provide energy for treatment of the sample. In some embodiments, (i) the first light source is operable to provide energy for treatment of the sample, (ii) the second light source is operable to provide energy for treatment of the sample, or (iii) both (i) and (ii). In some embodiments, (i) the first light source is one or more of a cauterizing source, a coagulating source, a cutting source, a calcifying source, and a heating source, (ii) the second light source is one or more of a cauterizing source, a coagulating source, a cutting source, a calcifying source, and a heating source, or both (i) and (ii). In some embodiments, (i) the first light source is operable to provide energy for controlling freezing of the sample, (ii) the second light source is operable to provide energy for controlling freezing of the sample, or (iii) both (i) and (ii).

In some aspects, the present disclosure provides a method for treating at least a portion of a sample, the method comprising providing energy for treating the at least a portion of the sample during a bright phase of a first light source, wherein the bright phase of the first light source occurs during a dark phase of a second light source. In some embodiments, the method comprises receiving, via a detector, a signal generated, at least in part, using second light provided by the second light source. In some embodiments, the signal is an optical coherence tomography (OCT) signal. In some embodiments, the signal is a near-infrared spectroscopy (NIRS) signal.

In some embodiments, a method comprises cutting at least a portion of a sample using energy provided by a light source. In some embodiments, a method comprises coagulating at least a portion of a sample using energy provided by a light source. In some embodiments, a method comprises heating at least a portion of a sample using energy provided by a light source. In some embodiments, a method comprises calcifying at least a portion of a sample using energy provided by a light source. In some embodiments, a method comprises controlling freezing of at least a portion of a sample using energy provided by a light source.

### BRIEF DESCRIPTION OF THE DRAWING

The drawing is presented herein for illustration purposes, not for limitation. The foregoing and other objects, aspects, features, and advantages of various embodiments will become more apparent and may be better understood by referring to the following description taken in conjunction with the accompanying drawing, in which:

FIG. 1A is a block diagram of a system (e.g., a sample characterization and/or treatment system) using a common probe for two light sources, according to illustrative embodiments;

FIG. 1B is a block diagram of a system (e.g., a sample characterization and/or treatment system) using distinct probes for each of two light sources, according to illustrative embodiments;

FIG. 1C is a schematic of a system (e.g., a sample characterization and/or treatment system) using a common probe for two light sources and a rotary junction, according to illustrative embodiments;

FIG. 1D is a block diagram of a system (e.g., a sample characterization and/or treatment system) using a common probe for two light sources, which is optically connected to two detectors, according to illustrative embodiments;

FIG. 1E is a block diagram of a system (e.g., a sample characterization and/or treatment system) using distinct probes for each of two light sources, each probe optically connected to a distinct detector, according to illustrative embodiments;

FIG. 2 is a flow diagram of an example method of characterizing a sample (e.g., a material in a sample), according to illustrative embodiments;

FIG. 3A is a plot of emission wavelength versus time for a light source, according to illustrative embodiments;

FIG. 3B is a plot of emission intensity versus time for a light source, according to illustrative embodiments;

FIG. 4A is a plot of emission wavelength versus time for two interleaved light sources, according to illustrative embodiments;

FIG. 4B is a plot of emission wavelength versus time for two interleaved light sources, according to illustrative embodiments;

FIG. 4C is a plot of emission intensity versus time for two interleaved light sources, according to illustrative embodiments;

FIG. 5A is a plot of emission wavelength versus time for a light source, according to illustrative embodiments;

FIG. 5B is a plot of emission wavelength versus time for two interleaved light sources, according to illustrative embodiments;

FIG. 6 is a plot of emission wavelength versus time for three interleaved light sources, according to illustrative embodiments; and

FIG. 7 is a flow diagram of an example method of treating a sample, according to illustrative embodiments.

### DEFINITIONS

In order for the present disclosure to be more readily understood, certain terms used herein are defined below. Additional definitions for the following terms and other terms may be set forth throughout the specification.

In this application, unless otherwise clear from context or otherwise explicitly stated, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Light source”: As used herein, a “light source” refers to a source that provides (e.g., emits) light. Light is electromagnetic radiation (EMR) (e.g., photons). Light may have a frequency in a visible spectrum or not. A light source may emit one or more of visible light, near-infrared light, infrared light, long wavelength infrared light, ultraviolet light, deep ultraviolet light, and extreme ultraviolet light. In some embodiments, a light source may emit terahertz radiation. A light source may emit x-rays, microwaves, or radio waves. A light source may be, but is not necessarily, a laser. A light source may be, for example, a light source with reduced temporal coherence such as a source comprising a light emitting diode (LED) or a superluminescent diode (SLD). A light source may be a swept source or a broadband source. In certain embodiments, a light source is a swept source. In certain embodiments, a light source is a swept-source laser. In certain embodiments, a light source is a broadband source. As used herein, “first” and “second” are arbitrary designations with respect to two light sources and may be interchanged in some embodiments. Moreover, any optional feature described with respect to a first light source may be applied to a second light source and vice versa.

“Bright phase” and “dark phase”: As used herein, a “bright phase” refers to a period of time during which a light source is providing light and a “dark phase” refers to a period of time during which a light source is not providing light. A light source alternates between a bright phase and a dark phase during operation. In certain embodiments, a light source is a swept source and at least a portion of the dark phase for the light source is a cycle phase. It is understood that, in practice, during a dark phase for a light source, some minimal amount of light may unintentionally escape from the light source [e.g., less than 1% (e.g., less than 0.1% or less than 0.01%) of the amount provided during a light phase for the light source].

“Cycle phase”: As used herein, “cycle phase” refers to a period of time during which a light source cycles back to an initial state in order to provide light across a wavelength range as intended at another future time period. For example, a swept source laser may cycle back from an ending wavelength to an initial wavelength during a cycle phase. A cycle phase of a light source may be shorter than or equal to the length of time of a dark phase of the light source. For example, a dark phase of a light source may be a cycle phase of the light source. For example, a dark phase of a light source may comprise a cycle phase and a delay period wherein the light source is neither cycling nor in a bright phase.

“Delay period”: As used herein, a “delay period” is a portion of a dark phase of a swept source that is not part of its cycle phase. A delay period is not a result of manufacturing tolerances; it is a period that a light source is intentionally arranged and constructed to have. For example, a system may be arranged and constructed as to have a light source with a dark phase, wherein, for a short delay period at the beginning or end (or both) of the dark phase, the light source is not cycling.

“Image”: As used herein, the term “image,” for example, as in a two- or three-dimensional image of tissue (or other sample), includes any visual representation, such as a photo, a video frame, streaming video, as well as any electronic, digital, or mathematical analogue of a photo, video frame, or streaming video. Any system or apparatus described herein, in certain embodiments, includes a display for displaying an image or any other result produced by a processor. Any method described herein, in certain embodiments, includes a step of displaying an image or any other result produced by the method. Any system or apparatus described herein, in certain embodiments, outputs an image to a remote receiving device [e.g., a cloud server, a remote monitor, or a hospital information system (e.g., a picture archiving and communication system (PACS))]. In some embodiments, an image is produced using a fluorescence imaging system, a spectroscopic imaging system, a luminescence imaging system, and/or a reflectance imaging system. In certain embodiments, a tomographic image and a spectroscopic image are co-registered to form a composite image. In some embodiments, an image is a two-dimensional (2D) image. In some embodiments, an image is a three-dimensional (3D) image. In some embodiments, an image is a reconstructed image. An image (e.g., a 3D image) may be a single image or a set of images. An imaging technique (e.g., using light provided by a light source) may produce one or more images.

“Probe”: As used herein, “probe” refers to a portion of a device or apparatus, or a subsystem, that directs light from one or more light sources toward a sample. Each light source in a system may have a distinct probe or two or more (e.g., all) light sources may share a common probe. A probe may comprise one or more optical elements, such as, for non-limiting examples, one or more lenses, one or more mirrors, and/or one or more waveguides (e.g., optical fibers). A probe may comprise any one or combination of one or more single mode fibers and one or more multi-mode fibers. For example, a probe may comprise one or more multi-clad fibers, such as a double clad fiber. A probe may comprise a housing (e.g., a sheath, for example, if the probe is part of a catheter).

“Sample”: As used herein, “sample” refers to a thing to be characterized. Generally, any material, mixture, substance, or capable of characterization by a light can be used as a sample. A sample may comprise one or more materials. A sample may be gaseous, fluid, or solid. A sample may be, for example, a gel (e.g., a hydrogel), an elastomer, or a composite. A sample may be a biological sample. For example, a sample may be an organ or biological structure (e.g., tissue) or portion thereof. A sample may be an in vivo organ or in vivo tissue. For example, a sample may be an in vivo artery or portion thereof. A sample may comprise one or more features of interest. For example, a feature of interest may be, for example, arterial plaque (e.g., a vulnerable plaque, for example having a fibrous cap).

### DETAILED DESCRIPTION OF CERTAIN EMBODIMENTS

Throughout the description, where articles, devices, and systems are described as having, including, or comprising specific components, or where processes and methods are described as having, including, or comprising specific steps, it is contemplated that, additionally, there are articles, devices, and systems of the present disclosure that consist essentially of, or consist of, the recited components, and that there are processes and methods according to the present disclosure that consist essentially of, or consist of, the recited processing steps. Where a component is described as being “arranged and constructed to” provide or perform (e.g., a function or step of a method), embodiments are contemplated where that component is programmed or otherwise settable to provide or perform (e.g., the function or step of the method). For example, a light source arranged and constructed to have a bright phase may be programmed to have a bright phase or may be set (e.g., using one or more physical controls) to have a bright phase. Headers are provided for the convenience of the reader and are not to be construed to be limiting with respect to the claimed subject matter.

It should be understood that the order of steps or order for performing certain action is immaterial so long as the method to which the steps or actions belong remains operable. Moreover, in some embodiments, two or more steps or actions (e.g., or portions thereof) may be conducted simultaneously.

Described herein are systems with interleaved light sources and methods of their use. For example, interleaved light sources may be used for sample characterization. Each of two or more interleaved light sources may be used to provide light to a sample (e.g., for characterization purposes). Light from each of a plurality of light sources may correspond to illumination light for a distinct characterization technique. For example, one light source may provide light for a imaging technique (e.g., a tomography technique) and one light source may provide light for a spectroscopy technique (e.g., near-infrared spectroscopy).

In some embodiments, a system comprises a plurality of light sources. For example, a system may comprise two light source or three light sources or more than three light sources. In some embodiments, a light source is a laser (e.g., a vertical-cavity laser or vertical-cavity surface-emitting laser). A light source may be a swept source or a broadband source. In some embodiments, a first light source is a swept source and a second light source is a broadband source. A light source may be tunable such that its emission wavelength or range of emission wavelengths can be changed (e.g., for samples with different characteristics). A light source emits light of one or more wavelengths. For example, a light source may emit or be operable to emit one wavelength at a time (e.g., is a swept source) or multiple wavelengths (e.g., a range of wavelengths) at a time (e.g., is a broadband source). A light source may be operable to emit multiple discrete wavelengths (e.g., not a range) at a time. A light source may be operable to emit multiple ranges (e.g., multiple discrete ranges) of light.

In some embodiments, a first light source is substantially similar to a second light source. A first light source substantially similar to a second light source emits light having one or more substantially similar (within manufacturing tolerances) emission characteristics to light emitted by the second light source. The one or more emission characteristics that are substantially similar may include one or more of emission wavelength(s), bandwidth, linewidth, emission intensity, emission pulse time, bright phase duration, and dark phase duration. In some embodiments, a first light source substantially similar to a second light source emits light that is identical (within manufacturing tolerances) to light emitted by the second light source. In some embodiments, a plurality of interleaved light sources are substantially similar while each light source is in optical communication with a distinct detector. For example, a first light source may be substantially identical to a second light source, where the first and second light sources are interleaved and the first light source is in optical communication with an OCT detector and a second light source is in optical communication with a NIRS detector.

A light source may emit visible light. A light source may emit light that is not visible (e.g., alternatively or additionally to emitting visible light), such as near-infrared light, infrared light, long wavelength infrared light, ultraviolet light, deep-ultraviolet light, or extreme ultraviolet light. A light source may emit x-rays. A light source may emit terahertz radiation. A light source may emit microwaves. A light source may emit radio waves. A light source may comprise a superluminescent diode or a light-emitting diode (LED). For example, a light source comprise be an LED with a peak emission wavelength in a visible, UV, deep UV, extreme UV, near-infrared, infrared, or long wavelength infrared spectrum. A light source may comprise an LED comprising one or more phosphors or one or more species of quantum dots (e.g., such that the LED emits white light).

A light source may provide (e.g., emit) light useful for a characterization technique. A characterization technique may use, at least in part, light from a light source to characterize a sample (e.g., structurally or chemically). For example, a signal may be generated using, at least in part, light from a light source. A signal may be generated, at least in part, using light reflected, refracted, scattered, transmitted (e.g., a non-absorbed portion of initially incident light), or produced [e.g., by photoluminescence (e.g., fluorescence, phosphorescence, or Raman emission), chemiluminescence, or bioluminescence)] by a sample in response to light from a light source impinging on the sample, for example. A signal may be an interference signal (e.g., using light received and/or generated from a sample and light from a reference arm). A signal generated using, at least in part, light from a light source may be detected by a detector, such as a camera or an interferometer for example. In some embodiments, a system may be constructed and arranged so that a detector only receives and processes signal during a period corresponding (e.g., temporally) to a bright phase of a particular light source (e.g., which is offset from, encompasses, or is coincident with the bright phase). Such a period may be controlled, for example, using one or more controllable optics.

A light source may provide (e.g., emit) light for one or more characterization techniques. For example, a light source may be a light source for an imaging technique (e.g., a tomography technique), a scattering technique, a diffraction technique, or a spectroscopy technique for example. A light source may provide light, for example, for UV-vis spectroscopy, infrared spectroscopy (e.g., near-infrared spectroscopy (NIRS)), photoluminescence spectroscopy, wavelength dispersive x-ray spectroscopy, x-ray photoelectron spectroscopy, or x-ray photo correlation spectroscopy. In some embodiments, a light source provides light for near-infrared spectroscopy. A light source may provide light, for example, for a 2D imaging technique or a 3D imaging technique (e.g., a tomography technique). A light source may provide light, for example, for an optical imaging technique or an x-ray imaging technique. An optical imaging technique may be, for example, fluorescence imaging, such as fluorescence tomography. An optical imaging technique may be, for example, optical coherence tomography (OCT) or diffuse optical imaging (e.g., diffuse optical tomography). A light source may provide light, for example, for an interferometry technique, such as an OCT technique, an optical frequency domain imaging technique, phase contract imaging, or differential interference contract microscopy for example. A light source may provide light, for example, for a phase imaging technique. A light source may provide light, for example, for x-ray diffraction, small-angle x-ray scattering, or wide-angle x-ray scattering. A system may include a first light source that provides light for an imaging technique and a second light source that provides light for a spectroscopy technique. A light source may be used to generate spatially resolved data (e.g., imaging and/or spectroscopy data). Interleaved light sources may provide an advantage (e.g., as compared to non-interleaved light sources), for example in that they facilitate providing accurate co-registration of spatially resolved data from a plurality of techniques (e.g., each performed using light from one light source).

In some embodiments, data from a first characterization technique (e.g., performed using a first light source) and data from a second characterization technique (e.g., performed using a second light source) are co-registered. Co-registered data may be temporally and/or spatially co-registered. Co-registered data may be collected using interleaved light sources. In some embodiments, interleaved light sources are used to provide accurately co-registered data due, at least in part, to data for each of a plurality of characterization techniques being collected at substantially similar locations and/or times during data collection (e.g., during scanning) as a result of the interleaving. For example, a catheter comprising a common probe for multiple characterization techniques, each utilizing one of a plurality of interleaved light sources, may collect accurately co-registered data due to the interleaving allowing collection of data from substantially similar locations at substantially similar times during pullback of the catheter.

A light source for a spectroscopy technique (e.g., NIRS) may be a swept source. A light source for an imaging technique (e.g., OCT) may be a swept source. A light source for a spectroscopy technique (e.g., NIRS) may be a broadband source. A light source for an imaging technique (e.g., OCT) may be a broadband source. In some embodiments, a first characterization technique (e.g., an imaging or) is performed using a first light source and a second characterization technique is performed using the first light source and a second light source that is interleaved with the first light source (e.g., in a catheter). In some embodiments, a first characterization and a second characterization technique are both performed using a first light source and a second light source that is interleaved with the first light source (e.g., in a catheter).

A light source may comprise (e.g., be) one or more of a laser, a superluminescent diode, a light-emitting diode, an incandescent source, a discharge source. A light source may be a laser. In some embodiments, a light source may be a vertical cavity laser (e.g., a vertical-cavity surface-emitting laser). In some embodiments, a light source is a vertical-cavity surface-emitting laser. A light source that is a laser may comprise a rod laser, a dye laser, and/or a gas laser. In some embodiments, a light source is or includes a fiber laser (e.g., is or includes an optical fiber laser). As an example, a fiber laser, in accordance with some embodiments, may be doped with one or more of erbium, ytterbium, neodymium, dysprosium, praseodymium, thulium, and holmium. A light source may be a Fabry-Perot laser, a distributed feedback (DFB) laser, or a distributed Bragg reflector (DBR) laser, for example. A light source may be a laser diode or a quantum cascade laser, for example. A light source may be an amplified spontaneous emission (ASE) light source. For example, a light source may be an ASE optical fiber (e.g., doped with one or more rare-earth metals).

A light source may be a single frequency light source. For example, a light source may have a linewidth of no more than 5 kHz (e.g., no more than 3 kHz, no more than 2 kHz, or no more than 1 kHz). For example, a light source that is a laser may be a single frequency laser (e.g., having a linewidth of no more than 5 kHz, no more than 3 kHz, no more than 2 kHz, or no more than 1 kHz). A light source may be a multi-mode light source. For example, light source (e.g., a laser) may emit light comprising a plurality of modes [e.g., at least 10 modes, at least 100 modes, at least 1,000 modes, at least 10,000 modes (e.g. and no more than 100,000 modes or no more than 50,000 modes)]. In some embodiments, a light source is a swept source laser having a linewidth of no more than 5 kHz that is swept over a range of at least 10 nm (e.g., at least 20 nm, at least 40 nm, at least 50 nm, at least 75 nm, at least 100 nm, at least 125 nm, at least 150 nm, or at least 200 nm) and no more than 500 nm (e.g., no more than 300 nm or no more than 200 nm).

In some embodiments, a light source is only operable to emit a single wavelength. It is understood that, in practice, light sources that emit one wavelength at a time (e.g., only a single wavelength), may have some emission bandwidth [e.g., of less than 3 nm, less than 2 nm, less than 1 nm, less than 0.5 nm, less than 0.4 nm, less than 0.2 nm, less than 0.1 nm, less than 0.5 nm, or less than 0.3 nm (e.g., as measured at full width at half maximum)]. A light source may have a bandwidth of less than 50 nm, less than 40 nm, less than 30 nm, less than 20 nm, less than 10 nm, or less than 5 nm (e.g., as measured at full width at half maximum) (e.g., if the light source comprises an LED).

In some embodiments, a light source is a broadband light source that emits light over a range of wavelengths. For example, a light source may be a broadband light source that emits light over a range of at least 20 nm, at least 40 nm, at least 50 nm, at least 60 nm, at least 80 nm, at least 100 nm, at least 150 nm, at least 200 nm, at least 300 nm, at least 400 nm, or at least 500 nm (e.g., and no more than 2000 nm, no more than 1000 nm, or no more than 500 nm). In some embodiments, a light source is a broadband source that emits light in a range of no more than 500 nm (e.g., no more than 300 nm, no more than 250 nm, no more than 200 nm, no more than 150 nm, no more than 100 nm, no more than 75 nm, or no more than 50 nm). In some embodiments, a light source emits light one wavelength at a time over a range of wavelengths (e.g., is swept over the range), wherein the range is at least 20 nm, at least 40 nm, at least 50 nm, at least 60 nm, at least 80 nm, at least 100 nm, at least 150 nm, at least 200 nm, at least 300 nm, at least 400 nm, or at least 500 nm (e.g., and no more than 2000 nm, no more than 1000 nm, or no more than 500 nm). A first light source and a second light source may each be a broadband light source where the respective broadband ranges at least partially overlap.

In some embodiments, a light source is a swept source that provides light (e.g., sweeps) within a range of no more than 500 nm (e.g., no more than 400 nm, no more than 300 nm, no more than 250 nm, no more than 200 nm, no more than 150 nm, no more than 125 nm, no more than 100 nm, no more than 75 nm, no more than 50 nm, no more than 40 nm, or no more than 25 nm) (e.g., and at least 75 nm or at least 100 nm). In some embodiments, a light source is a swept source that provides light (e.g., sweeps) sweeps within a range of at least 50 nm (e.g., at least 75 nm, at least 100 nm, at least 125 nm, at least 150 nm, at least 200 nm, at least 250 nm, or at least 300 nm) (e.g., and no more than 500 nm). In some embodiments, a light source is a swept source (e.g., a swept source laser) that provides light (e.g., sweeps) within a range of no more than 150 nm. In some embodiments, a light source is a swept source (e.g., a swept source laser) that provides light (e.g., sweeps) within a range of no more than 100 nm. In some embodiments, a first light source is a swept source laser that provides light (e.g., sweeps) within a range of no more than 200 nm (e.g., no more than 150 nm or no more than 100 nm) and a second light source is a swept source laser that provides light (e.g., sweeps) within a range of no more than 200 nm (e.g., no more than 150 nm or no more than 100 nm). A first light source and a second light source may each be a swept source where the respective sweeping ranges at least partially overlap. In some embodiments, a light source may emit light in a narrow band that is swept over a larger range. For example, a light source may emit light in a band of no more than 5 nm, no more than 10 nm, no more than 15 nm, no more than 20 nm, no more than 30 nm, no more than 50 nm and no more than 100 nm, swept over a larger range of, for example, no more than 20 nm, no more than 30 nm, no more than 50 nm, no more than 100 nm, no more than 200 nm, no more than 300 nm, no more than 400 nm, or no more than 500 nm. A range may be centered around a wavelength, for example in a UV, visible, near-infrared, or infrared spectrum.

A light source may be operable to emit light centered at or around a desired wavelength. For example, a desired wavelength may correspond to a characterization peak of a sample being characterized (e.g., a constituent material thereof). A light source may be operable to emit light within a range of a central emission wavelength. For example, a light source may be operable to emit light having a wavelength, or wavelengths, within 10 nm of a central emission wavelength, within 20 nm of a central emission wavelength, within 50 nm of a central emission wavelength, within 100 nm of a central emission wavelength, within 150 nm of a central emission wavelength, or within 200 nm of a central emission wavelength. It should be understood that “within” in this context means in either direction (higher and lower) of the central emission wavelength. A light source may be a swept source that sweeps over a range of wavelengths centered around a central emission wavelength (e.g., that is approximately a characterization peak for a sample). A light source may be a broadband source that simultaneously emits light of a number of (e.g., all) wavelengths within a range centered around a central emission wavelength.

In some embodiments, a light source is a broadband source that includes (e.g., that is approximately centered around) a characterization peak for a sample. In some embodiments, a light source is a swept source that includes (e.g., that is approximately centered around) a characterization peak for a sample. In some embodiments, a characterization peak is in a range from about 1100 nm to about 1400 nm. For example, a characterization peak may be about 1300 nm (e.g., in a range from about 1280 to 1320 nm) or about 1200 nm (e.g., in a range from about 1180 to about 1220 nm). In some embodiments, a light source is a swept source that emits light within a band centered around a central emission wavelength where the central emission wavelength is in a range from about 1100 nm to about 1400 nm (e.g., and where the band has a range of, for example, no more than 200 nm, no more than 300 nm, no more than 500 nm, or no more than 700 nm).

In certain embodiments, each of a plurality of light sources provides light that is used in a characterization technique to characterize a biological sample. A biological sample may have a characterization peak corresponding to a wavelength in an ultraviolet, visible, or infrared spectrum. For example, a plurality of light sources may be used to provide light in a cardiac catheter. Accordingly, a characterization peak for a biological sample correspond to collagen, elastin, plaque (e.g., lipid rich plaque), or cholesterol. A biological sample characterized using light from a plurality of light sources may comprise one or more of collagen, elastin, plaque (e.g., lipid rich plaque), and cholesterol. Such constituents have characterization peaks that are generally known and, in some examples, are in a range from about 1100 nm to about 1400 nm in wavelength. Without wishing to be bound by any particular theory, collagen, elastin, plaque (e.g., lipid rich plaque), and cholesterol can be identified and/or differentiated using one or more techniques using light having a wavelength in a range from about 1100 nm to about 1400 nm [e.g., from a light source that is a broadband or swept source covering this range (e.g., interleaved with a second light source providing similar wavelengths of light or a subset thereof)]. In some embodiments, a characterization peak for a biological sample is a characterization peak for cholesterol in the biological sample corresponding to a wavelength of about 1300 nm (e.g., in a range from about 1280 nm to about 1320 nm). In some embodiments, arterial plaque may be characterized using interleaved light sources that each emit light corresponding to one or more characterization peaks (e.g., the same or different peak(s) for each light source) for the arterial plaque. In some embodiments, a characterization technique may be performed using light having a wavelength corresponding to a characterization peak for characterizing arterial plaque to characterize (e.g., identify and/or differentiate) one or more molecules in a region around a plaque deposit. A characterization peak for characterizing arterial plaque may be a characterization peak of collagen, elastin, plaque (e.g., lipid rich plaque), or cholesterol for example.

A light source has a dark phase and a bright phase. During a dark phase, a light source is not (e.g., intentionally) providing light. During a bright phase, a light source is providing light. During operation of a system, each of a plurality of light sources alternates between a dark phase and a bright phase of the light source (e.g., during a period). In some embodiments, a system may comprise or be used with a probe that spatially scans over a sample and a light source may alternate between a dark phase and a bright phase throughout scanning. A light source may alternate between a bright phase and a dark phase during operation at a rate of at least 10 Hz, at least 100 Hz, at least 1 kHz, at least 2 kHz, at least 5 kHz, at least 10 kHz, at least 15 kHz, at least 20 kHz, at least 50 kHz, at least 75 kHz, or at least 100 kHz (e.g., and no more than 10 GHz, no more than 5 GHz, no more than 2 GHz, no more than 1 GHz, no more than 500 MHz, no more than 250 MHz, no more than 100 MHz, no more than 50 MHz, no more than 10 MHz, no more than 1 MHz, no more than 500 kHz, no more than 250 kHz, no more than 100 kHz). In some embodiments, a light source may alternate between a bright phase and a dark phase at a frequency that corresponds (e.g., proportionately) to a rotational frequency of a probe, for example in a catheter, such as a cardiac catheter. If a light source is a swept source, the light source may be scanned across its wavelength range during a bright phase.

A bright phase and a dark phase of a light source may be equal or unequal in length of time. In some embodiments, a light source has a duty cycle greater than 50% (e.g., greater than 60%, greater than 70%, greater than 80%). In some embodiments, a light source has a duty cycle less than 50% (e.g., less than 40%, less than 33%, less than 30%, less than 25%, less than 20%, less than 15%, less than 10%, less than 7%, less than 5%, less than 3%, or less than 1%). In some embodiments, a light source has a duty cycle of about 50%. In some embodiments, a first light source has a duty cycle of less than 50% (e.g., less than 40%, less than 30%, less than 25%, less than 20%, less than 15%, less than 10%, less than 7%, less than 5%, less than 3%, or less than 1%) and a second light source interleaved with the first light source has a duty cycle of less than 50% (e.g., less than 40%, less than 30%, less than 25%, less than 20%, less than 15%, less than 10%, less than 7%, less than 5%, less than 3%, or less than 1%). In some embodiments, a first light source has a duty cycle of less than 50% (e.g., less than 40%, less than 30%, less than 25%, less than 20%, less than 15%, less than 10%, less than 7%, less than 5%, less than 3%, or less than 1%) and a second light source interleaved with the first light source has a duty cycle of greater than 50% (e.g., greater than 60%, greater than 70%, greater than 80%). In some embodiments, a first interleaved light source has a duty cycle less than 33%, a second interleaved light source has a duty cycle less than 33%, and a third interleaved light source has a duty cycle less than 33%. A first light source may have a different duty cycle than a second light source (e.g., which may also be different from a duty cycle of a third light source).

A bright phase of a first light source occurs during a dark phase of a second light source (i.e., the first light source and the second light source are interleaved). In some embodiments, a bright phase of the second light source occurs during a dark phase of the first light source. In some embodiments, a bright phase of the second light source occurs at least partially (e.g., not entirely) during a dark phase of the first light source. A bright phase of a first light source may be coincident with a dark phase of a second light source and/or a dark phase of a first light source may be coincident with a bright phase of a second light source. A bright phase of a first light source may be shorter than a dark phase of a second light source. A bright phase of a second light source may be shorter than a dark phase of a second light source. As used herein, a first phase or period that occurs “during” a second phase or period may occur, for example, throughout the second phase or period or during only a portion of the second phase or period (e.g., when the second phase or period is longer than the first phase or period).

When a light source is a swept source, a dark phase of the light source may include a cycle phase. For example, one or more (e.g., two, three, more than three, or all) light sources in a system may be swept sources that each include a cycle phase during their dark phase. A dark phase of a light source may be a cycle phase. When two or more light sources are each swept sources, their cycle phases may not be of equal length of time. During a cycle phase, a swept source can reset from an ending wavelength of its sweep to an initial wavelength, such that the swept source is again in an initial state, ready to be swept again. An ending wavelength may be higher or lower than an initial wavelength. In some embodiments, a swept source is constructed and arranged to sweep from only one initial wavelength to one final wavelength (i.e., the initial and final wavelengths are not settable). In some embodiments, a swept source has a settable initial and final wavelength of a sweep. A dark phase of a swept source may be a cycle phase of the swept source. That is, in some embodiments, a swept source emits light during operation only when it is not cycling (e.g., back to its initial wavelength).

In some embodiments, a swept source includes one or more delay periods and a cycle phase. For example, a first light source may have a bright phase that is longer than a cycle phase of a second light source such that a dark phase of the second light source includes a delay period (e.g., making the bright phase of the first light source and the dark phase of the second light source equal in length of time). A delay period may be shorter than, equal to, or longer than a length of a cycle phase of a swept source. A delay period may occur before, after, or during a cycle phase in a dark phase of a light source.

A light source may provide variable intensity of light during a bright phase of the light source. For example, intensity may vary versus time or intensity may vary with each repetition of the bright phase. Intensity of light provided by a light source may be wavelength dependent. Intensity of light provided by a light source may be constant (e.g., within manufacturing tolerances) during a bright phase of the light source. A light source may alternate between fixed intensities (e.g., of a single or multiple wavelengths) as it alternates between bright and dark phases. In some embodiments, a light source is controllable to emit light of different fixed (e.g., constant) intensity during a bright phase. In some embodiments, a light source is controllable to emit light of different variable intensities during a bright phase.

A light source may emit light with constant intensity during a bright phase of the light source. A light source may emit light in a pulse. A light source may emit a variable intensity of light during a bright phase.

In some embodiments, a light source may be a source constructed and arranged to provide light to alter at least a portion of a sample in at least one way. In some embodiments, a system comprises a plurality of interleaved light sources comprising a first light source, a second light source, and a third light source, where the first and second light sources each provide light for a characterization technique (e.g., an imaging technique for one and a spectroscopy technique for the other) to characterize a sample and the third light source provides light to alter at least a portion of the sample in at least one way (e.g., contemporaneously with characterization). For example, when used in a cardiac catheter, a first two light sources may be constructed and arranged to provide light for use in identifying plaque in a coronary artery in need of treatment (e.g., wherein one of the two light sources is an imaging source, such as an OCT imaging source, and one is a spectroscopy source, such as a NIRS source) and, if present, a third light source may be constructed and arranged to treat the plaque. Treatment of a plaque may include reducing a size of the plaque, or alteration of its contents to stabilize the plaque (e.g., thereby reducing its potential for rupture). In some embodiments, a system comprises a plurality of interleaved light sources comprising a first light source and a second light source, where the first light source provides light for a characterization technique to characterize a sample and the second light source provides light to alter at least a portion of the sample in at least one way (e.g., contemporaneously with characterization).

In some embodiments, altering at least a portion of a sample in at least one way, for example treating (e.g., a plaque or other tissue), can be accomplished, at least in part, by, for example, one or more of cutting, heating, freezing, coagulating, cauterizing, and calcifying with energy provided from light from a light source during a bright phase (e.g., that is interleaved with one or more other light sources). A cauterizing source is a source that emits light able to cauterize at least a portion of a sample (e.g., tissue) (e.g., due to the light's wavelength and/or intensity). A cutting source is a source that emits light able to cut at least a portion of a sample (e.g., tissue) (e.g., due to the light's wavelength and/or intensity). A calcifying source is a source that emits light able to calcify at least a portion of a sample (e.g., a plaque deposit in an artery) (e.g., due to the light's wavelength and/or intensity). A coagulating source is a source that emits light able to coagulate at least a portion of a sample (e.g., a plaque deposit in an artery) (e.g., due to the light's wavelength and/or intensity). In some embodiments, freezing may be controlled, at least in part, using light from a light source (e.g., that is interleaved with one or more other light sources) (e.g., and accomplished using one or more additional freezing elements, for example in a catheter). In some embodiments, application of a treatment, e.g. at least in part with light from a third light source, to a location identified by two techniques, each using light from one of a first light source and a second light source, may be improved by reduced co-registration error between images (e.g., co-registered optical and spectroscopic images) generated by the two techniques.

A light source that provides energy for treating a sample may alternate between a bright phase and a dark phase at a frequency that corresponds (e.g., is integrally proportional) to a rotational frequency of a probe used to transmit (e.g., in a catheter, such as a cardiac catheter). For example, a light source that provides energy for treating a sample may alternate between a bright phase and a dark phase during operation at a rate of at least 10 Hz, at least 100 Hz, at least 1 kHz, at least 2 kHz, at least 5 kHz, at least 10 kHz, at least 15 kHz, at least 20 kHz, at least 50 kHz, at least 75 kHz, or at least 100 kHz (e.g., and no more than 10 GHz, no more than 5 GHz, no more than 2 GHz, no more than 1 GHz, no more than 500 MHz, no more than 250 MHz, no more than 100 MHz, no more than 50 MHz, no more than 10 MHz, no more than 1 MHz, no more than 500 kHz, no more than 250 kHz, no more than 100 kHz).

In some embodiments, a system comprises a plurality of light sources comprising a first light source, a second light source, and a third light source, where a dark phase of the first light source and a dark phase of the second light source are at least partially coincident, defining a common dark period for the first and second light sources. In some such embodiments, a bright phase of the third light source occurs during the common dark period for the first and second light sources. Some such embodiments may facilitate contemporaneous characterization (e.g., OCT imaging and/or NIRS characterization) during treatment, for example during cutting, coagulating, cauterizing, calcifying, heating, or controlling of freezing (e.g., thereby improving quality and/or accuracy and and/or precision of the cut, coagulation, cauterization, calcification, heating, or controlled freezing). For example, contemporaneous characterization during treating (e.g., using any one of the preceding examples of treatments) may improve what is effectively a co-registration of treatment location and characterization location. A third light source may be a used to provide light for a characterization technique (e.g., a third characterization technique).

A system may include one or more probes. A probe may include one or more optics. Optics may be used to, for example, alter an optical path of light from a light source, collect light from a sample, or filter illumination or sample light. Example optics include lenses (e.g., ball lenses), one or more mirrors, and/or one or more waveguides (e.g., optical fibers). A probe may include an optical bench to hold and position one or more optics relative to an optical path of incoming light from a light source and/or an optical path of sample light received by the probe. A system may include a rotary junction, for example when used with (e.g., when comprising) a probe in a catheter system. A rotary junction may be arranged and constructed to transmit light from a first light source and a second light source to a common probe that is freely rotatable. A probe may include a housing (e.g., a sheath) that houses one or more waveguides (e.g., fiber optics). In some embodiments, a system comprises, or is arranged and constructed to be connected to (e.g., comprising one or more optical connections or junctions for), a distinct probe for each light source. In some embodiments, a system comprises, or is arranged and constructed to be connected to (e.g., comprising one or more optical connections or junctions for), a common probe for at least two light sources in a plurality of light sources in the system. A common probe may include a plurality of waveguides. For example each of a plurality of waveguides may correspond to one of a plurality of light sources. A waveguide in a common probe may be arranged and constructed to transmit a signal to a detector. A plurality of waveguides may include a core and a waveguide cladding of a multi-clad fiber or a plurality of independent optical fibers.

A probe may include one or more waveguides for transmitting light from one or more light sources to a sample. One or more waveguides may be one or more optical fibers, such as one or more single mode fibers and/or one or more multi-mode fibers. In some embodiments, a probe comprises a multi-clad fiber (e.g., a double clad fiber) wherein light from one light source is provided through a core of the fiber and light from a second light source is provided through a cladding of the fiber. In certain embodiments, a probe comprises a multi-clad fiber and light from a plurality of light sources is provided through a core of the fiber and a sample signal is received through a cladding (or vice versa).

A system may be a catheter. A catheter may be, for example, a cardiac catheter. In some embodiments, a catheter comprises a probe and a plurality of light sources comprising a first light source and a second light source. A catheter including a plurality of interleaved light sources may provide accurately co-registered data for a plurality of characterization techniques that is collected during catheter pullback. In some embodiments, the first and second light sources are arranged and constructed so that the first light source has a dark phase and the second light source has a bright phase that occurs during the dark phase of the first light source. In some embodiments, a bright phase of the first light source occurs during a dark phase of the second light source. In some embodiments, a catheter comprising a probe and two light sources, each having a bright phase and a dark phase, the bright phase of one of the two light sources occurring during the dark phase of the other of the two light sources, can allow for a reduced rotational frequency of the probe in order to produce images (e.g., for subsequent analysis). In some embodiments, a reduced rotational frequency of a probe can produce smoother pullback. In some embodiments, a catheter comprising a probe and two light sources, each having a bright phase and a dark phase, the bright phase of one of the two light sources occurring during the dark phase of the other of the two light sources, can allow for increased data collection at a given pullback rate (as compared to a catheter using non-interleaved light sources).

Illustrative Light Sources, Systems Including the Same, and Methods of Their Use

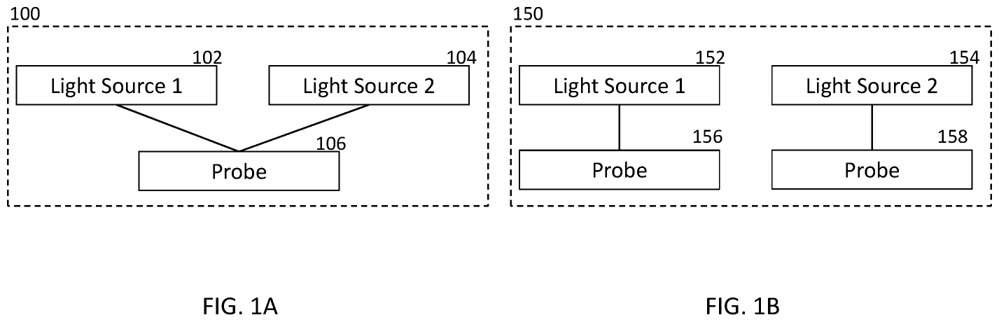

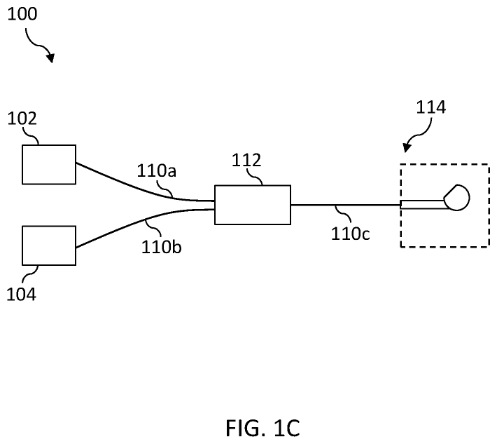

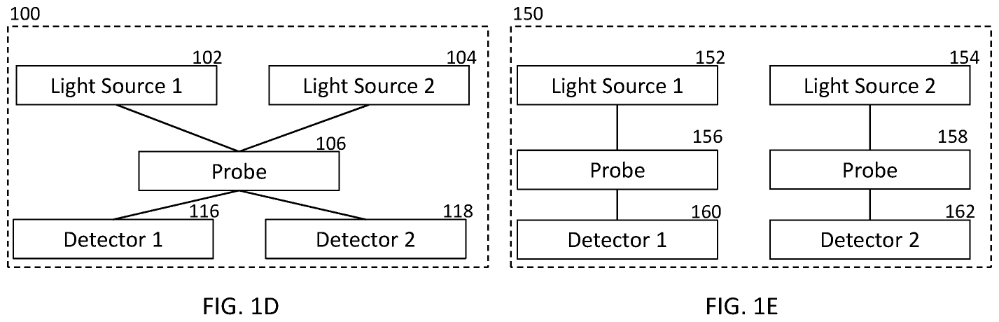

FIG. 1A is a block diagram of an example system 100. An example system 100 comprises a first light source 102, a second light source 104, and a common probe 106. Example system 100 is arranged and constructed such that a first light source 102 and a second light source 104 are optically connected to a common probe 106. FIG. 1B is a block diagram of example system 150. An example system 150 comprises a first light source 152 optically connected to a distinct first probe 156 and a second light source 154 optically connected to a distinct second probe 158.

Optical connections are indicated by solid lines between blocks in FIGS. 1A and 1B. An optical connection may include one or more waveguides (e.g., optical fibers), for example. One or more intermediate optical elements maybe included in an optical connection, such as one or more mirrors, one or more lenses, one or more splitters, one or more combiners, one or more filters, and/or one or more, for example.

An example system 100 may be a catheter. For example, FIG. 1C shows a simple illustrative embodiment where the example system 100 is a catheter. A first light source 102 is optically connected to a first waveguide 110a and a second light source 104 is optically connected to a second waveguide 110b. The first waveguide 110a and the second waveguide 110b are connected to a rotary junction 112. The first waveguide 110a and the second waveguide 110b may be coupled (e.g., spliced) into a common waveguide (e.g., a multi-clad fiber) before rotary junction 112 (not shown). A probe 114 includes a third waveguide 110c that is optically connected to the rotary junction 112. (The portion of the probe 114 in the dashed box is magnified for clarity and includes a modified ball lens used to redirect light towards a sample, for example towards a wall of a lumen, such as an artery.) The third waveguide 110c transmits light from first light source 102 and second light source 104. In some embodiments, the example system 100 is arranged such that light from the first light source 102 travels along a core of the third waveguide 110c and light from the second light source 104 travels along a waveguide cladding of the third waveguide 110c, or vice versa. Similarly, example system 100 may be arranged, in some embodiments, such that the core of the third waveguide 110c receives and transmits received light (e.g., a sample signal) to a first detector and the waveguide cladding of the third waveguide 110c receives and transmits received light (e.g., a sample signal) to a second detector. In some embodiments, the rotary junction 112 is omitted and the first and second waveguides 110a-b are optically and physically connected to (e.g., spliced into) the third waveguide 110c. Operation of light sources may be begin once pullback of the catheter is initiated or a short period of time before pullback is initiated).

FIG. 1D is a block diagram of an example system 100. An example system 100 comprises a first light source 102, a second light source 104, a common probe 106, a first detector 116, and a second detector 118. Example system 100 is arranged and constructed such that a first light source 102 and a second light source 104 are optically connected to a common probe 106. A signal generated using, at least in part, light from the first light source 102 is detected by the first detector 116. A signal generated using, at least in part, light from the second light source 104 is detected by the second detector 118. The first detector 116 may be constructed and arranged to detect received light (e.g., a signal), for example, at least during a portion of the bright phase of the first light source 102 (e.g., only during the bright phase). The second detector 118 may be constructed and arranged to detect received light (e.g., a signal), for example, at least during a portion of the bright phase of the second light source 104 (e.g., only during the bright phase). The bright phase of the first light source 102 may occur during the dark phase of the second light source 104 and, optionally, vice versa. Other characteristics that the first light source 102 and second light source 104 may optionally have are described throughout this disclosure.

FIG. 1E is a block diagram of example system 150. An example system 150 comprises a first light source 152 optically connected to a distinct first probe 156, which is optically connected to a first detector 160, and a second light source 154 optically connected to a distinct second probe 158, which is optically connected to a second detector 162. The first detector 160 may be constructed and arranged to detect received light (e.g., a signal), for example, at least during a portion of the bright phase of the first light source 152 (e.g., only during the bright phase). The second detector 162 may be constructed and arranged to detect received light (e.g., a signal), for example, at least during a portion of the bright phase of the second light source 154 (e.g., only during the bright phase). The bright phase of the first light source 152 may occur during the dark phase of the second light source 154 and, optionally, vice versa. Other characteristics that the first light source 152 and second light source 154 may optionally have are described throughout this disclosure.

Optical connections are indicated by solid lines between blocks in FIGS. 1D and 1E. An optical connection may include one or more waveguides (e.g., optical fibers), for example. One or more intermediate optical elements maybe included in an optical connection, such as one or more mirrors, one or more lenses, one or more splitters, one or more combiners, one or more filters, and/or one or more, for example.

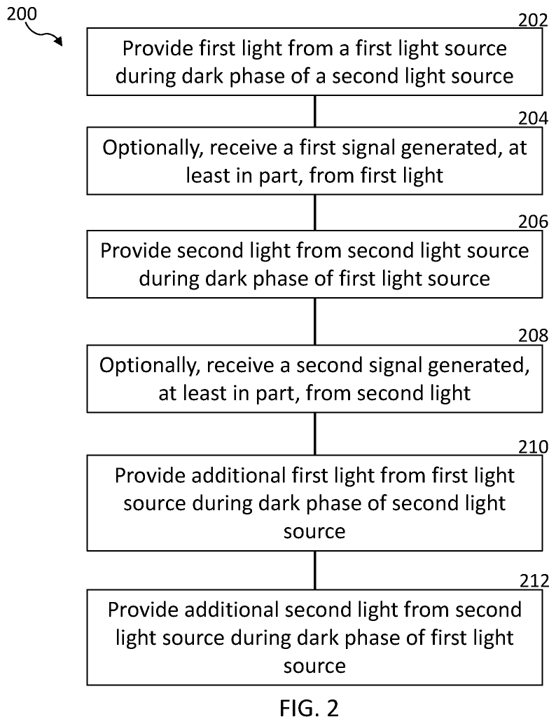

FIG. 2 is an example method 200 of characterizing a sample. The example method 200 includes steps 202-212 (where step 204 and step 208 are optional). In step 202, first light from a first light source is provided during a dark phase of a second light source. In optional step 204, a first sample signal generated, at least in part, using the first light is received (e.g., by a first detector, such as a photodetector or a CCD or CMOS camera for example). Optional step 204 may occur during a bright phase of the first light source. For example, a first light source may be pulsed during a bright phase and a sample signal is received during the bright phase. Optional step 204 may occur during the dark phase of the second light source. In step 206, second light from a second light source is provided during a dark phase of the first light source. In optional step 208, a second sample signal generated, at least in part, using the second light is received (e.g., by a second detector, such as a photodetector or a CCD or CMOS camera for example). The first detector and the second detector may be the same detector. Optional step 208 may occur during a bright phase of the second light source. For example, a second light source may be pulsed during a bright phase and a sample signal is received during the bright phase. Optional step 208 may occur during the dark phase of the first light source. In step 210, additional first light from the first light source is provided during the dark phase of the second light source. In step 212, additional second light from the second light source is provided during the dark phase of the first light source.

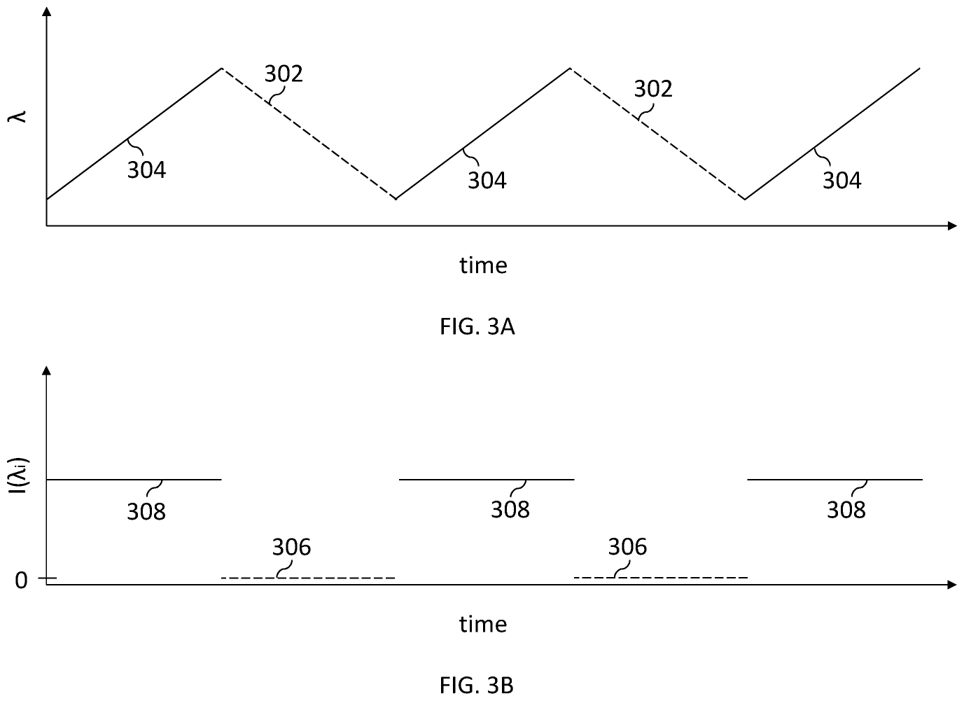

FIG. 3A shows a plot of wavelength versus time for an exemplary light source, with a bright phase 304 and a dark phase 302, that is a swept source. The light source alternates between its bright phase 304 and its dark phase 302 during operation. The exemplary light source is a swept source that emits light of one wavelength at a time during its bright phase 304, swept within a range during operation. The dark phase 302 of the exemplary light source is a cycle phase where the exemplary light source cycles from an ending wavelength to an initial wavelength. In operation, the exemplary light source provides light over a range from an initial wavelength to an ending wavelength and then cycles back to the initial wavelength and then provides light again. For simplicity, the exemplary light source is shown to have a duty cycle of 50%, but shorter duty cycles are also contemplated.

FIG. 3B shows a plot of intensity (which may be wavelength dependent) versus time for an exemplary light source, with a bright phase 308 and a dark phase 306, that is a broadband source. During the bright phase 308, the exemplary light source provides light of multiple wavelengths at a time (e.g., over a range of wavelengths). During the dark phase 306, the exemplary light source emits no light. The light source alternates between its bright phase 308 and its dark phase 306 during operation. Although shown as a constant non-zero intensity during the bright phase 308, it is understood that this may represent an average intensity (e.g., if pulsed) or may be a true constant intensity. For simplicity, the exemplary light source is shown to have a duty cycle of 50%, but shorter duty cycles are also contemplated.

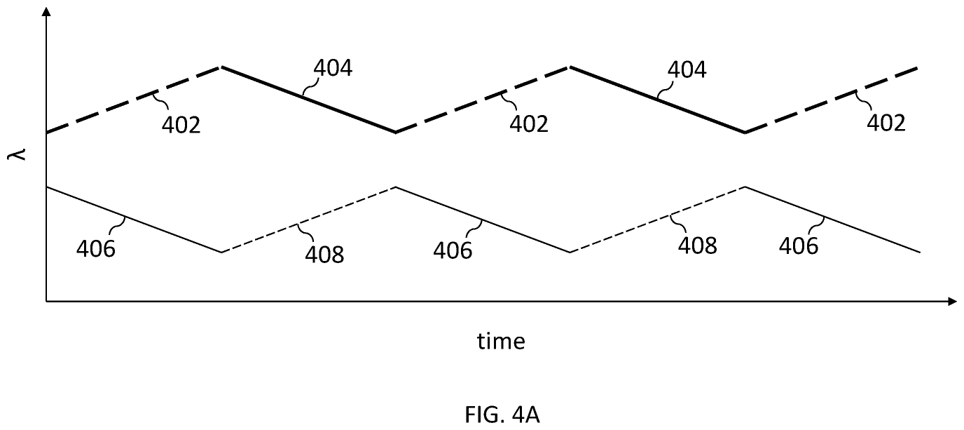

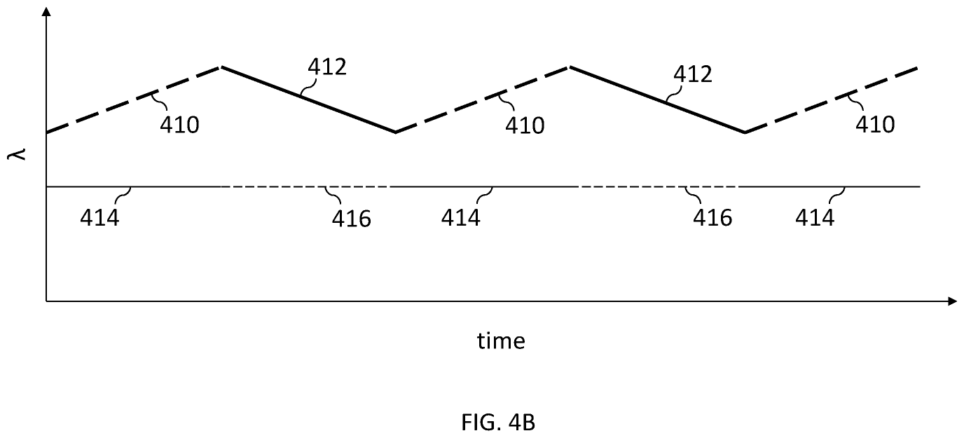

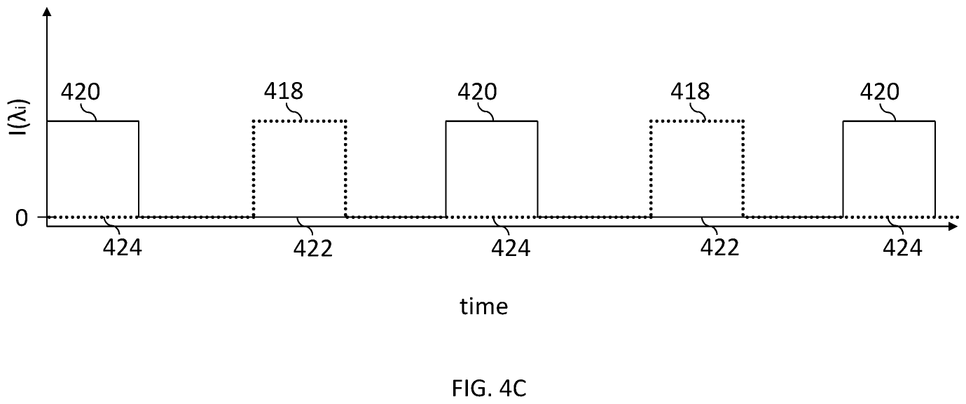

FIG. 4A shows a plot of wavelength versus time for an exemplary system comprising two light sources: a first light source and a second light source. The first light source emits light within a wavelength range that is higher than a wavelength range in which the second light source emits light. The first light source has a bright phase 404 and a dark phase 402. The second light source has a bright phase 406 and a dark phase 408. The first light source and the second light source are each a swept source. Moreover, the dark phase 402 of the first light source is coincident with the bright phase 406 of the second light source and the bright phase 404 of the first light source is coincident with the dark phase 408 of the second light source. The dark phase 402 of the first light source and the dark phase 408 of the second light source are each a cycle phase of the respective light source. The first light source alternates between its bright phase 404 and its dark phase 402 during operation. The second light source alternates between its bright phase 406 and its dark phase 408 during operation.

FIG. 4B shows a plot of wavelength versus time for an exemplary system comprising two light sources: a first light source and a second light source. The first light source emits light within a wavelength range that is higher than a wavelength of light emitted by the second light source. The first light source has a bright phase 412 and a dark phase 410. The second light source has a bright phase 406 and a dark phase 408. The first light source is a swept source. The second light source emits light of a single wavelength. Although shown as emitting a single wavelength, it is understood that in some similar embodiments, the second source emits light of a range of wavelengths including (e.g., centered around) the plotted wavelength (e.g., even overlapping with wavelengths emitted by the first light source). The dark phase 410 of the first light source is coincident with the bright phase 414 of the second light source and the bright phase 412 of the first light source is coincident with the dark phase 416 of the second light source. The dark phase 410 of the first light source is a cycle phase. The first light source alternates between its bright phase 412 and its dark phase 410 during operation. The second light source alternates between its bright phase 414 and its dark phase 416 during operation. The second light source may be a light source that provides light for characterizing a sample or may be a light source that provides light for altering (e.g., treating) at least a portion of a sample in at least one way (e.g., cutting, cauterizing, calcifying, coagulating, heating, and/or controlling freezing).

FIG. 4C shows a plot of intensity versus time for an exemplary system that includes a pair of interleaved light sources. A first light source has a bright phase 418 and a dark phase 424. A second light source has a bright phase 420 and dark phase 422. The bright phase 418 of the first light source occurs during the dark phase 422 of the second light source. The bright phase 420 of the first light source occurs during the dark phase 424 of the second light source. Both the first light source and the second light source have a duty cycle of less than 50%. In this example, for purposes of illustration, the intensity versus time for both the first light source and the second light source is a square wave. In some embodiments, intensity as a function of time during a bright phase of a light source (e.g., in a pair or triplet of interleaved light sources) is not constant (e.g., a plot of intensity versus time is not a square wave).

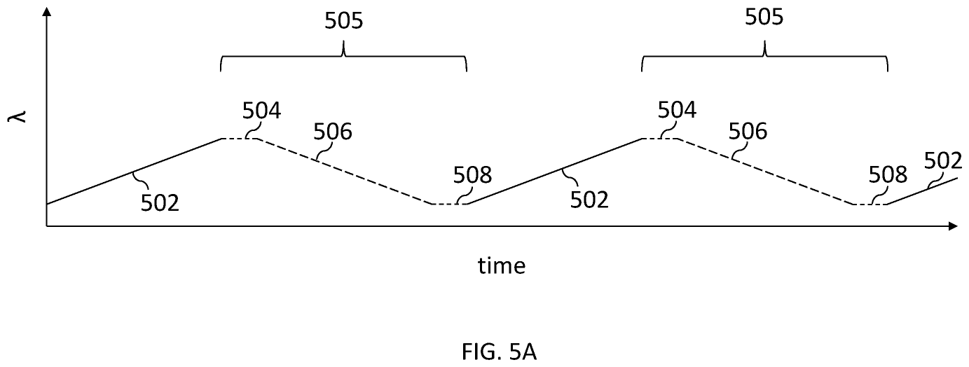

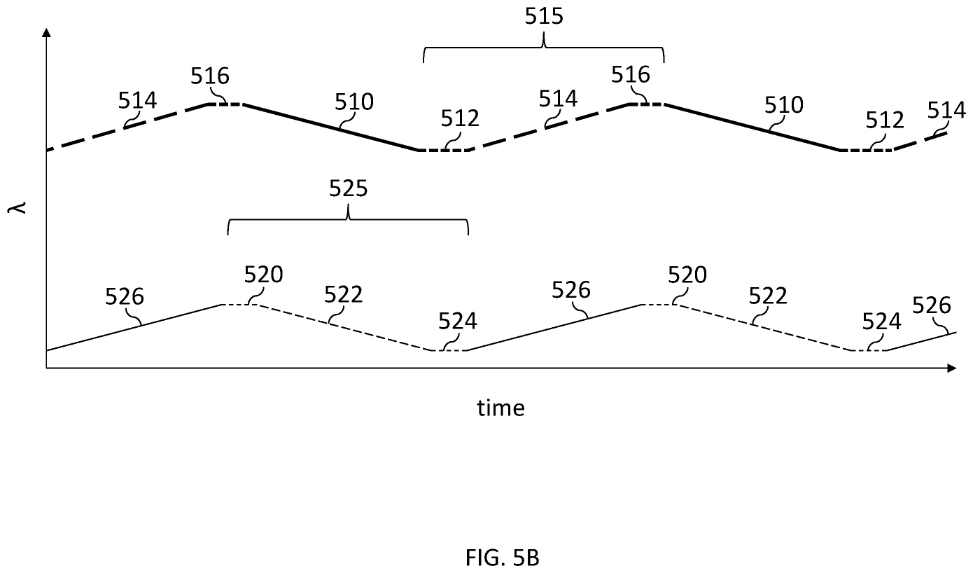

FIG. 5A shows a plot of wavelength versus time for an exemplary light source. The light source has a bright phase 502 and a dark phase 505. The dark phase 505 includes a first delay period 504, a cycle phase 506, and a second delay period 508. During the delay periods, the exemplary light source is not cycling. The exemplary first light source alternates between its bright phase 502 and its dark phase 505 during operation.

FIG. 5B shows a plot of wavelength versus time for an exemplary system comprising two light sources: a first light source and a second light source. A first light source has a bright phase 510 and a dark phase 515. The dark phase 515 of the first light source comprises a first delay period 512, a cycle phase 514, and a second delay period 516. Only one repetition of the bright phase 510 and the dark phase 515 of the first light source are labeled. A second light source has a bright phase 526 and a dark phase 525. The dark phase 525 of the second light source comprises a first delay period 520, a cycle phase 522, and a second delay period 524. Only one repetition of the bright phase 526 and the dark phase 525 of the second light source are labeled. Due to the relative lengths of the various periods, the dark phase 515 of the first light source is partially coincident with the dark phase 525 of the second light source, defining a common dark period.

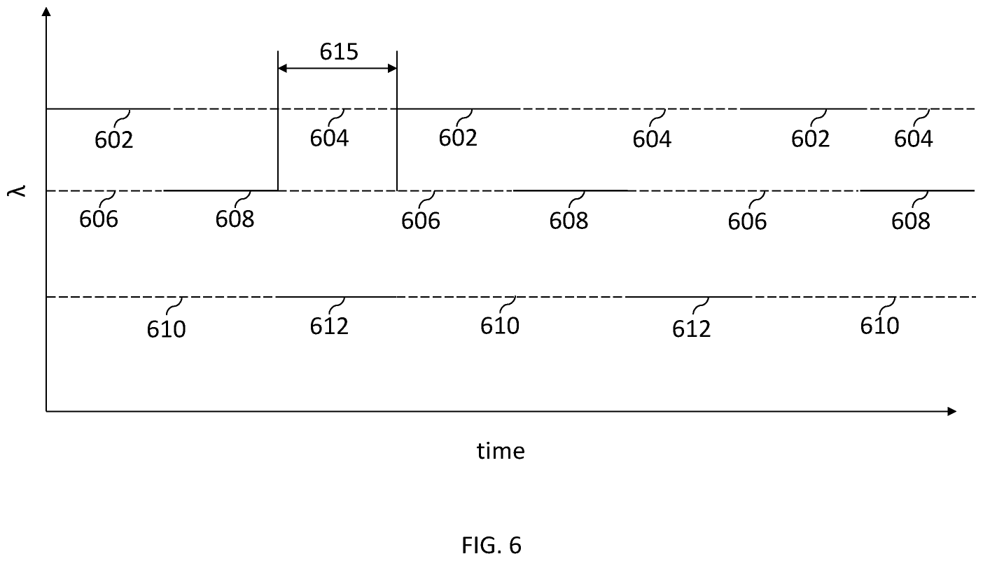

FIG. 6 shows a plot of wavelength versus time for an exemplary system comprising three light sources: a first light source, a second light source, and a third light source. Each of the first, second, and third light sources emits a single wavelength or a range of wavelengths (e.g., overlapping) that includes (e.g., is centered around) the shown wavelength. The first light source has a bright phase 602 and a dark phase 604. The second light source has a bright phase 608 and a dark phase 606. The third light source has a bright phase 612 and a dark phase 610. The dark phase 604 of the first light source and the dark phase 606 of the second light source are coincident during a common dark period 615. The dark phase 604 of the first light source and the dark phase 606 of the second light source are partially coincident, defining a common dark period for the two light sources. The bright phase 612 of the third light source occurs during the common dark period of the first and second light sources. The bright phase 612 of the third light source is shown to occur throughout the common dark period due to the lengths of the various phases. Light from any one or more of the first, second, and third light sources may be used to provide energy to a sample to treat the sample (e.g., to calcify, cauterize, coagulate, heat, cut, or control freezing at least a portion of the sample). Light from any one or more of the first, second, and third light sources may be used to perform a characterization technique (e.g., a distinct characterization technique for each of the light sources). As shown, the bright phases of the first, second, and third light sources are exactly one third the length of their respective dark phases. This relationship occurs only for illustration purposes and systems disclosed herein are not limited to such relationships.

Combining the illustrative dark phase and/or bright phase of any first or second or third light source as described in relation to any one of FIGS. 3A-6 with the illustrative dark phase and/or bright phase of any first or second or third light source as described in relation to any one of FIGS. 3A-6 forms an illustrative embodiment. Combining a first or second or third light source as described in relation to any one of FIGS. 3A-6 with a first or second or third light source as described in any one of FIGS. 3A-6 form an illustrative embodiment.

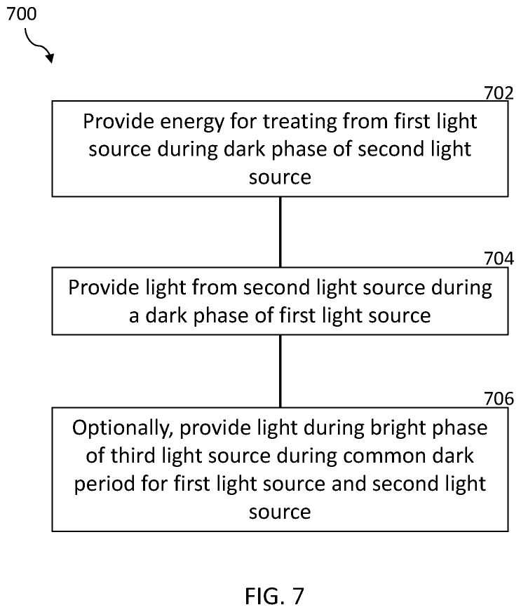

FIG. 7 is a flow diagram of example method 700 for treating at least a portion of a sample, the method 700 comprising step 702, step 704, and optional step 706. In step 702, energy (e.g., from light) for treating (e.g., by cutting, calcifying, cauterizing, coagulating, heating, or controlling freezing) at least a portion of a sample is provided by a first light source during a bright phase of the first light source, which occurs during a dark phase of a second light source. In step 704, light from the second light source is provided (during a bright phase of the second light source) during a dark phase of the first light source. In optional step 706, light is provided by the third light source (during a bright phase of the third light source) during a common dark period for the first light source and the second light source.

Certain embodiments of the present application were described above. It is, however, expressly noted that the present application is not limited to those embodiments, but rather the intention is that additions and modifications to what was expressly described in the present application are also included within the scope of the application. Moreover, it is to be understood that the features of the various embodiments described in the present application were not mutually exclusive and can exist in various combinations and permutations, even if such combinations or permutations were not made express, without departing from the spirit and scope of the application. Having described certain implementations of systems and methods for interleaving light sources, it will now become apparent to one of skill in the art that other implementations incorporating the concepts of the application may be used. Therefore, the application should not be limited to certain implementations, but rather should be limited only by the spirit and scope of the following claims.

What is claimed is:

1. A system comprising: a plurality of light sources, each of which has a bright phase and a dark phase, the system being arranged and constructed so that, for a first light source and a second light source of the plurality of light sources, the bright phase of the first light source occurs during the dark phase of the second light source.

2. The system of claim 1, wherein the system is arranged and constructed so that the bright phase of the second light source occurs during the dark phase of the first light source.

3. The system of claim 2, wherein the system is arranged and constructed so that the bright phase of the first light source is coincident with the dark phase of the second light source and the dark phase of the first light source is coincident with the bright phase of the second light source.

4. The system of any one of the preceding claims, wherein the first light source is a swept source (e.g., a swept source laser) (e.g., that emits near-infrared light).

5. The system of claim 4, wherein the dark phase for the first light source comprises a cycle phase.

6. The system of claim 5, wherein the dark phase for the first light source is the cycle phase.

7. The system of any one of the preceding claims, wherein the second light source is a swept source (e.g., a swept source laser) (e.g., that emits near-infrared light).

8. The system of claim 7, wherein the swept source is constructed and arranged to emit light in a wavelength band comprising (e.g., centered around) a characterization peak for characterizing arterial plaque (e.g., by sweeping through the band).

9. The system of claim 8, wherein the characterization peak is about 1300 nm (e.g., is in a range of about 1280 nm to about 1320 nm).

10. The system of any one of claims 7-9, wherein the swept source is operable to sweep within a second light source wavelength band, wherein the second light source wavelength band has a range of no more than 300 nm (e.g., wherein a central emission wavelength of the second light source wavelength band is in a range from about 1100 nm to about 1400 nm).

11. The system of claim 10, wherein the wavelength band has a range of no more than 150 nm.

12. The system of any one of the preceding claims, wherein the first light source is a swept source laser operable to emit light in a first light source wavelength band having a range of no more than 200 nm (e.g., no more than 125 nm) (e.g., wherein a central emission wavelength of the first light source wavelength band of the first light source is in a range from about 1100 nm to about 1400 nm).

13. The system of claim 12, wherein the first light source wavelength band comprises (e.g., is centered around) a first source characterization peak for characterizing arterial plaque (e.g., wherein the first source characterization peak is about 1200 nm).

14. The system of any one of claims 1-3 and 7-11, wherein the first light source is a broadband source (e.g., and the second light source is a broadband source) [e.g., and the second light source is a swept source (e.g., a swept source laser) (e.g., that emits near-infrared light)].

15. The system of claim 14, wherein the broadband source emits light having a plurality of wavelengths within a range of 150 nm (e.g., including one or more characterization peaks for characterizing arterial plaque).

16. The system of claim 15, wherein the one or more characterization peaks includes a peak of about 1200 nm.

17. The system of any one of the preceding claims, the system comprises a common probe for the first light source and the second light source, the common probe comprising an optical fiber.

18. The system of claim 17, wherein the system is operable to (i) provide the first light source via a cladding of the optical fiber and the second light source via a core of the optical fiber, (ii) provide the second light source via a cladding of the optical fiber and the first light source via a core of the optical fiber, or (iii) provide both the first light source and the second light source via a core of the optical fiber.

19. The system of claim 17 or claim 18, comprising a rotary junction arranged and constructed to transmit light from the first light source and the second light source to the common probe, wherein the common probe is freely rotatable.

20. The system of any one of the preceding claims, wherein the system comprises an optical coherence tomography (OCT) subsystem comprising an OCT detector and the second light source.

21. The system of any one of the preceding claims, wherein the system comprises a near-infrared spectroscopy (NIRS) subsystem comprising a NIRS detector and the first light source.

22. The system of any one of the preceding claims, wherein the system is arranged and constructed to be operable as a cardiac catheter.

23. The system of any one of the preceding claims, wherein the system is constructed and arranged so that (i) alternation between the bright phase and the dark phase of the first light source occurs at a first light source alternation frequency of at least 10 Hz, (ii) alternation between the bright phase and the dark phase of the second light source occurs at a second light source alternation frequency of at least 10 Hz, or (iii) both (i) and (ii).

24. The system of claim 23, wherein the first light source alternation frequency is at least 10 kHz and the second light source alternation frequency is at least 10 kHz.

25. The system of any one of the preceding claims, wherein the plurality of light sources comprises a third light source, wherein the system is arranged and constructed so that the dark phase of the first light source and the dark phase of the second light source are partially coincident, defining a common dark period for the first light source and the second light source, and the bright phase of the third light source occurs during the common dark period.

26. The system of any one of the preceding claims, comprising a first detector arranged and constructed to detect a first signal corresponding to a first characterization technique and a second detector arranged and constructed to detect a second signal corresponding to a second characterization technique, wherein the first signal is generated using, at least in part, light from the first light source, and the second signal is generated using, at least in part, light from the second light source.

27. The system of claim 26, wherein the first detector is an OCT detector and the second detector is a NIRS detector.

28. The system of any one of the preceding claims, wherein the first light source and the second light source are substantially similar.

29. A method comprising providing (e.g., emitting) first light (e.g., to a sample) from a first light source during a dark phase of a second light source.

30. The method of claim 29, further comprising providing (e.g., emitting) second light (e.g., to the sample) from the second light source during a dark phase of the first light source.

31. The method of claim 29 or claim 30, wherein the first light source provides the first light throughout the dark phase of the second light source.

32. The method of claim 30 or claim 31, wherein the second light source provides the second light throughout the dark phase of the first light source.

33. The method of any one of claims 29-32, comprising cycling the second light source during the dark phase of the second light source.

34. The method of any one of claims 30-33, comprising cycling the first light source during the dark phase of the first light source.

35. The method of any one of claims 29-34, comprising receiving, via a first detector, a first signal generated, at least in part, using the first light.

36. The method of claim 35, wherein the first signal is an optical coherence tomography (OCT) signal.

37. The method of any one of claims 30-36, comprising receiving, via a second detector, a second signal generated, at least in part, using the second light.

38. The method of claim 37, wherein the second signal is a near-infrared spectroscopy (NIRS) signal.

39. The method of any one of claims 29-38, comprising transmitting the first light through a common probe for the first light source and the second light source.

40. The method of claim 39, comprising rotating the common probe (e.g., wherein the common probe is in optical communication with a rotary junction).

41. The method of claims 39 and 40, wherein a catheter (e.g., a cardiac catheter) comprises the first light source and the second light source and the common probe.

42. The method of any one of claims 29-41, wherein the first light is used to perform imaging (e.g., OCT).

43. The method of any one of claims 29-42, wherein the second light is used to perform spectroscopy (e.g., NIRS).

44. The method of any one of claims 29-43, comprising providing (e.g., emitting) third light from a third light source during a dark phase for the first light source and the dark phase for the second light source.

45. The method of any one of claims 29-44, wherein the first light source is a swept source (e.g., a swept source laser).

46. The method of any one of claims 29-44, wherein the first light source is a broadband source.

47. The method of any one of claims 29-46, wherein the second light source is a swept source (e.g., a swept source laser).

48. The method of any one of claims 29-46, wherein the second light source is a broadband source.

49. The method of any one of claims 29-48, wherein the method is performed using the system of any one of claims 1-28.

50. A system for characterizing a sample, the system comprising: a plurality of light sources, each of which has a bright phase and a dark phase, the system being arranged and constructed so that, for a first light source and a second light source of the plurality of light sources, the bright phase of the first light source occurs during the dark phase of the second light source, wherein each light source is for providing light for a characterization technique.

51. The system of claim 50, wherein the system is arranged and constructed so that the bright phase of the second light source occurs during the dark phase of the first light source.

52. The system of claim 51, wherein the system is arranged and constructed so that the bright phase of the first light source is coincident with the dark phase of the second light source and the dark phase of the first light source is coincident with the bright phase of the second light source.

53. The system of any one of claims 50-52, wherein the first light source is a swept source (e.g., a swept source laser) (e.g., that emits near-infrared light).

54. The system of claim 53, wherein the dark phase for the first light source comprises a cycle phase.

55. The system of claim 54, wherein the dark phase for the first light source is the cycle phase.

56. The system of any one of claims 50-55, wherein the second light source is a swept source (e.g., a swept source laser) (e.g., that emits near-infrared light).

57. The system of claim 56, wherein the swept source is constructed and arranged to emit light in a wavelength band comprising (e.g., centered around) a characterization peak for characterizing arterial plaque (e.g., by sweeping through the band).

58. The system of claim 57, wherein the characterization peak is about 1300 nm (e.g., is in a range from about 1280 nm to about 1320 nm).

59. The system of any one of claims 56-58, wherein the swept source is operable to sweep within a second light source wavelength band, wherein the second light source wavelength band has a range of no more than 300 nm (e.g., wherein a central emission wavelength of the second light source wavelength band is in a range from about 1100 nm to about 1400 nm).

60. The system of claim 59, wherein the wavelength band has a range of no more than 150 nm.

61. The system of any one of claims 50-60, wherein the first light source is a swept source laser operable to emit light in a first light source wavelength band having a range of no more than 200 nm (e.g., no more than 125 nm) (e.g., wherein a central emission wavelength of the first light source wavelength band of the first light source is in a range from about 1100 nm to about 1400 nm).

62. The system of claim 61, wherein the first light source wavelength band comprises (e.g., is centered around) a first source characterization peak for characterizing arterial plaque (e.g., wherein the first source characterization peak is about 1200 nm).

63. The system of any one of claims 50-52 and 56-60, wherein the first light source is a broadband source (e.g., and the second light source is a broadband source) [e.g., and the second light source is a swept source (e.g., a swept source laser) (e.g., that emits near-infrared light)].

64. The system of claim 63, wherein the broadband source emits light having a plurality of wavelengths within a range of 150 nm (e.g., including one or more characterization peaks for characterizing arterial plaque).

65. The system of claim 64, wherein the one or more characterization peaks includes a peak of about 1200 nm.

66. The system of any one of claims 50-65, the system comprises a common probe for the first light source and the second light source, the common probe comprising an optical fiber.

67. The system of claim 66, wherein the system is operable to (i) provide the first light source via a cladding of the optical fiber and the second light source via a core of the optical fiber, (ii) provide the second light source via a cladding of the optical fiber and the first light source via a core of the optical fiber, or (iii) provide both the first light source and the second light source via a core of the optical fiber.

68. The system of claim 66 or claim 67, comprising a rotary junction arranged and constructed to transmit light from the first light source and the second light source to the common probe, wherein the common probe is freely rotatable.

69. The system of any one of claims 50-68, wherein the system comprises an optical coherence tomography (OCT) subsystem comprising an OCT detector and the second light source.

70. The system of any one of claims 50-69, wherein the system comprises a near-infrared spectroscopy (NIRS) subsystem comprising a NIRS detector and the first light source.

71. The system of any one of claims 50-70, wherein the system is arranged and constructed to be operable as a cardiac catheter.

72. The system of any one of claims 50-71, wherein the system is constructed and arranged so that (i) alternation between the bright phase and the dark phase of the first light source occurs at a first light source alternation frequency of at least 10 Hz, (ii) alternation between the bright phase and the dark phase of the second light source occurs at a second light source alternation frequency of at least 10 Hz, or (iii) both (i) and (ii).

73. The system of claim 72, wherein the first light source alternation frequency is at least 10 kHz and the second light source alternation frequency is at least 10 kHz.

74. The system of any one of claims 50-73, wherein the plurality of light sources comprises a third light source, wherein the system is arranged and constructed so that the dark phase of the first light source and the dark phase of the second light source are partially coincident, defining a common dark period for the first light source and the second light source, and the bright phase of the third light source occurs during the common dark period.

75. The system of any one of claims 50-74, comprising a first detector arranged and constructed to detect a first signal corresponding to a first characterization technique and a second detector arranged and constructed to detect a second signal corresponding to a second characterization technique, wherein the first signal is generated using, at least in part, light from the first light source, and the second signal is generated using, at least in part, light from the second light source.

76. The system of claim 75, wherein the first detector is an OCT detector and the second detector is a NIRS detector.

77. The system of any one of claims 50-76, wherein the first light source and the second light source are substantially similar.

78. A method for characterizing a sample, the method comprising providing (e.g., emitting) first light to a sample from a first light source during a dark phase of a second light source, wherein light provided from the first light source is used in a first characterization technique and light provided from the second light source is used in a second characterization technique.

79. The method of claim 78, further comprising providing (e.g., emitting) second light (e.g., to the sample) from the second light source during a dark phase of the first light source.

80. The method of claim 78 or claim 79, wherein the first light source provides the first light throughout the dark phase of the second light source.

81. The method of claim 79 or claim 80, wherein the second light source provides the second light throughout the dark phase of the first light source.

82. The method of any one of claims 78-81, comprising cycling the second light source during the dark phase of the second light source.

83. The method of any one of claims 80-82, comprising cycling the first light source during the dark phase of the first light source.

84. The method of any one of claims 78-83, comprising receiving, via a first detector, a first signal generated, at least in part, using the first light.

85. The method of claim 84, wherein the first signal is an optical coherence tomography (OCT) signal.

86. The method of any one of claims 79-85, comprising receiving, via a second detector, a second signal generated, at least in part, using the second light.

87. The method of claim 86, wherein the second signal is a near-infrared spectroscopy (NIRS) signal.

88. A system for treating a sample, the system comprising: a plurality of light sources, each of which has a bright phase and a dark phase, the system being arranged and constructed so that, for a first light source and a second light source of the plurality of light sources, the bright phase of the first light source occurs during the dark phase of the second light source, wherein at least one of the plurality of light sources is operable to provide energy for treatment of the sample (e.g., and at least one of the first and second light sources is for providing light for a characterization technique).

89. The system of claim 88, wherein (i) the first light source is operable to provide energy for treatment of the sample, (ii) the second light source is operable to provide energy for treatment of the sample, or (iii) both (i) and (ii).

90. The system of claim 89, wherein (i) the first light source is one or more of a cauterizing source, a coagulating source, a cutting source, a calcifying source, and a heating source, (ii) the second light source is one or more of a cauterizing source, a coagulating source, a cutting source, a calcifying source, and a heating source, or both (i) and (ii).

91. The system of claim 89, wherein (i) the first light source is operable to provide energy for controlling freezing of the sample, (ii) the second light source is operable to provide energy for controlling freezing of the sample, or (iii) both (i) and (ii).

92. The system of any one of claims 88-91, wherein the system is arranged and constructed so that the bright phase of the second light source occurs during the dark phase of the first light source.

93. The system of claim 92, wherein the system is arranged and constructed so that the bright phase of the first light source is coincident with the dark phase of the second light source and the dark phase of the first light source is coincident with the bright phase of the second light source.

94. The system of any one of claims 88-93, wherein the first light source is a swept source (e.g., a swept source laser) (e.g., that emits near-infrared light).

95. The system of claim 94, wherein the dark phase for the first light source comprises a cycle phase (e.g., a swept source laser) (e.g., that emits near-infrared light).

96. The system of claim 95, wherein the dark phase for the first light source is the cycle phase.

97. The system of any one of claims 88-96, wherein the second light source is a swept source (e.g., a swept source laser) (e.g., that emits near-infrared light).

98. The system of claim 97, wherein the swept source is constructed and arranged to emit light in a wavelength band comprising (e.g., centered around) a characterization peak for characterizing arterial plaque (e.g., by sweeping through the band).

99. The system of claim 98, wherein the characterization peak is about 1300 nm (e.g., is in a range from about 1280 nm to about 1320 nm).

100. The system of any one of claims 97-99, wherein the swept source is operable to sweep within a second light source wavelength band, wherein the second light source wavelength band has a range of no more than 300 nm (e.g., wherein a central emission wavelength of the second light source wavelength band is in a range from about 1100 nm to about 1400 nm).

101. The system of claim 100, wherein the wavelength band has a range of no more than 150 nm.

102. The system of any one of claims 88-101, wherein the first light source is a swept source laser operable to emit light in a first light source wavelength band having a range of no more than 200 nm (e.g., no more than 125 nm) (e.g., wherein a central emission wavelength of the first light source wavelength band of the first light source is in a range from about 1100 nm to about 1400 nm).

103. The system of claim 102, wherein the first light source wavelength band comprises (e.g., is centered around) a first source characterization peak for characterizing arterial plaque (e.g., wherein the first source characterization peak is about 1200 nm).

104. The system of any one of claims 88-93 and 97-101, wherein the first light source is a broadband source (e.g., and the second light source is a broadband source) [e.g., and the second light source is a swept source (e.g., a swept source laser) (e.g., that emits near-infrared light)].

105. The system of claim 106, wherein the broadband source emits light having a plurality of wavelengths within a range of 150 nm (e.g., including one or more characterization peaks for characterizing arterial plaque).

106. The system of claim 105, wherein the one or more characterization peaks includes a peak of about 1200 nm.

107. The system of any one of claims 88-106, the system comprises a common probe for the first light source and the second light source, the common probe comprising an optical fiber.

108. The system of claim 107, wherein the system is operable to (i) provide the first light source via a cladding of the optical fiber and the second light source via a core of the optical fiber, (ii) provide the second light source via a cladding of the optical fiber and the first light source via a core of the optical fiber, or (iii) provide both the first light source and the second light source via a core of the optical fiber.

109. The system of claim 107 or claim 108, comprising a rotary junction arranged and constructed to transmit light from the first light source and the second light source to the common probe, wherein the common probe is freely rotatable.

110. The system of any one of claims 88-109, wherein the system comprises an optical coherence tomography (OCT) subsystem comprising an OCT detector and the second light source.

111. The system of any one of claims 88-110, wherein the system comprises a near-infrared spectroscopy (NIRS) subsystem comprising a NIRS detector and the first light source.

112. The system of any one of claims 88-111, wherein the system is arranged and constructed to be operable as a cardiac catheter.

113. The system of any one of claims 88-112, wherein the system is constructed and arranged so that (i) alternation between the bright phase and the dark phase of the first light source occurs at a first light source alternation frequency of at least 10 Hz, (ii) alternation between the bright phase and the dark phase of the second light source occurs at a second light source alternation frequency of at least 10 Hz, or (iii) both (i) and (ii).

114. The system of claim 113, wherein the first light source alternation frequency is at least 10 kHz and the second light source alternation frequency is at least 10 kHz.

115. The system of any one of claims 88-114, wherein the plurality of light sources comprises a third light source, wherein the system is arranged and constructed so that the dark phase of the first light source and the dark phase of the second light source are partially coincident, defining a common dark period for the first light source and the second light source, and the bright phase of the third light source occurs during the common dark period.

116. The system of claim 115, wherein the third light source is operable to provide energy for treatment of the sample.

117. The system of claim 116, wherein the third light source is one or more of a cauterizing source, a coagulating source, a cutting source, a calcifying source, and a heating source.

118. The system of claim 116, wherein the third light source is operable to provide energy for controlling freezing of the sample.

119. The system of any one of claims 88-118, comprising a first detector arranged and constructed to detect a first signal corresponding to a first characterization technique and a second detector arranged and constructed to detect a second signal corresponding to a second characterization technique, wherein the first signal is generated using, at least in part, light from the first light source, and the second signal is generated using, at least in part, light from the second light source.

120. The system of claim 119, wherein the first detector is an OCT detector and the second detector is a NIRS detector.

121. The system of any one of claims 88-120, wherein the first light source and the second light source are substantially similar.

122. A method for treating at least a portion of a sample, the method comprising providing energy for treating the at least a portion of the sample during a bright phase of a first light source, wherein the bright phase of the first light source occurs during a dark phase of a second light source.

123. The method of claim 122, comprising cutting the at least a portion of the sample using the energy.

124. The method of claim 122, comprising cauterizing the at least a portion of the sample using the energy.

125. The method of claim 122, comprising coagulating the at least a portion of the sample using the energy.

126. The method of claim 122, comprising heating the at least a portion of the sample using the energy.

127. The method of claim 122, comprising calcifying the at least a portion of the sample using the energy.

128. The method of claim 122, comprising controlling freezing of the at least a portion of the sample using the energy.

129. The method of any one of claims 122-128, further comprising providing (e.g., emitting) second light (e.g., to the sample) from the second light source during a dark phase of the first light source.

130. The method of any one of claims 122-129, wherein the first light source provides first light throughout the dark phase of the second light source.

131. The method of claim 129 or 130, wherein the second light source provides second light throughout the dark phase of the first light source.

132. The method of any one of claims 122-131, comprising receiving, via a detector, a signal generated, at least in part, using second light provided by the second light source.

133. The method of claim 132, wherein the signal is an optical coherence tomography (OCT) signal.

134. The method of claim 132, wherein the signal is a near-infrared spectroscopy (NIRS) signal.

135. The method of any one of claims 122-134, comprising (i) alternating the first light source between the bright phase and a dark phase of the first light source at a first light source alternation frequency of at least 10 Hz, (ii) alternating the second light source between a bright phase and the dark phase of the second light source at a second light source alternation frequency of at least 10 Hz, or (iii) both (i) and (ii).

136. The system of claim 135, wherein the first light source alternation frequency is at least 10 kHz and the second light source alternation frequency is at least 10 kHz.

137. The method of any one of claims 122-136, comprising providing (e.g., emitting) third light from a third light source during a dark phase for the first light source and the dark phase for the second light source.

138. The method of claim 138, comprising receiving, via a detector, a signal generated, at least in part, using third light provided by the third light source.

139. The method of claim 138, wherein the signal is an optical coherence tomography (OCT) signal.

140. The method of claim 138, wherein the signal is a near-infrared spectroscopy (NIRS) signal.
